(19) United States
(12) Patent Application Publication
(10) Pub. No.: US 2017/0333451 A1
(43) Pub. Date: Nov. 23, 2017

(54) DIHYDROTESTOSTERONE AND DIHYDROTESTOSTERONE DERIVATIVES AND PROMOTERS IN THE TREATMENT OF CANCER

(71) Applicant: Tyme, Inc., Wilmington, DE (US)

(72) Inventor: Steven Hoffman, Mahwah, NJ (US)

(21) Appl. No.: 15/598,818

(22) Filed: May 18, 2017

Related U.S. Application Data
(60) Provisional application No. 62/338,122, filed on May 18, 2016.

(51) Int. Cl. A61K 31/58; A61K 38/31; A61K 38/12; A61K 31/55; A61K 31/436; A61K 31/4166; A61K 31/407; A61K 31/37; A61K 31/198; A61K 45/06; A61K 31/19

(52) U.S. Cl. CPC A61K 31/58; A61K 45/06; A61K 38/31; A61K 38/12; A61K 31/55; A61K 31/19; A61K 31/4166; A61K 31/407; A61K 31/37; A61K 31/198; A61K 31/436

(57) ABSTRACT

The present disclosure is directed to methods of treating cancer comprising administering dihydrotestosterone, a dihydrotestosterone derivative, a dihydrotestosterone promoter, or a combination thereof to a patient in need of treatment.

### CROSS REFERENCE TO RELATED APPLICATIONS

This application claims the benefit of U.S. Provisional No. 62/338,122 filed May 18, 2016, the entirety of which is incorporated by reference herein.

### TECHNICAL FIELD

The present disclosure is directed to methods of treating cancer comprising administering dihydrotestosterone, a dihydrotestosterone derivative, a dihydrotestosterone promoter, or a combination thereof to a patient in need of treatment.

### BACKGROUND

According to the U.S. National Cancer Institute's Surveillance Epidemiology and End Results (SEER) database for the year 2008, nearly 12 million Americans have invasive cancers. Cancer is the second most common cause of death in the United States, behind only heart disease, and accounts for one in four deaths. It has been estimated that approximately 1600 Americans die of cancer each day. In addition to the medical, emotional and psychological costs of cancer, cancer has significant financial costs to both the individual and society. It is estimated by the National Institutes of Health that the overall costs of cancer in 2010 was $263.8 billion. In addition, it is estimated that another $140.1 billion is lost in productivity due to premature death.

Cancer treatments today include surgery, hormone therapy, radiation, chemotherapy, immunotherapy, targeted therapy, and combinations thereof. Surgical removal of cancer has advanced significantly; however, there remains a high chance of recurrence of the disease. Hormone therapy using drugs such as aromatase inhibitors and luteinizing hormone-releasing hormone analogs and inhibitors has been relatively effective in treating prostate and breast cancers. Radiation and the related techniques of conformal proton beam radiation therapy, stereotactic radiosurgery, stereotactic radiation therapy, intraoperative radiation therapy, chemical modifiers, and radio sensitizers are effective at killing cancerous cells, but can also kill and alter surrounding normal tissue. Chemotherapy drugs such as aminopterin, cisplatin, methotrexate, doxorubicin, daunorubicin and others alone and in combinations are effective at killing cancer cells, often by altering the DNA replication process. Biological response modifier (BRM) therapy, biologic therapy, biotherapy, or immunotherapy alter cancer cell growth or influence the natural immune response, and involve administering biologic agents to a patient such as an interferons, interleukins, and other cytokines and antibodies such as rituximab and trastuzumab and even cancer vaccines such as Sipuleucel-T.

Recently, new targeted therapies have been developed to fight cancer. These targeted therapies differ from chemotherapy because chemotherapy works by killing both cancerous and normal cells, with greater effects on the cancerous cells. Targeted therapies work by influencing the processes that control growth, division, and the spread of cancer cells and signals that cause cancer cells to die naturally. One type of targeted therapy includes growth signal inhibitors such as trastuzumab, gefitinib, imatinib, centuximab, dasatinib and nilotinib. Another type of targeted therapy includes angiogenesis inhibitors such as bevacizumab that inhibit cancers from increasing surrounding vasculature and blood supply. Yet another type of targeted therapy includes apoptosis-inducing drugs that are able to induce direct cancer cell death.

Although all of these treatments have been effective to one degree or another, they all have drawbacks and limitations. In addition to many of the treatments being expensive, they also are often too imprecise or the cancers are able to adapt to them and become resistant.

Thus, there is a great need for additional cancer treatments. In particular, there is a need for treatments for cancers that have become resistant to other forms of treatment.

### SUMMARY

The present disclosure is directed to methods of treating cancer in a patient comprising administering to the patient an effective amount of dihydrotestosterone, a dihydrotestosterone derivative, a dihydrotestosterone promoter, or a combination thereof.

### BRIEF DESCRIPTION OF THE FIGURES

FIG. 1 is an X-ray depicting a cancerous tumor on the distal radius of a canine.

FIG. 2 is an X-ray depicting calcification of the cancerous tumor shown in FIG. 1 after treatment according to a preferred aspect of the disclosure.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural form, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer. This condition, disease, or disorder can also be a symptom or side-effect of cancer.

As employed above and throughout the disclosure the term “effective amount” or “therapeutically effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds (including the described promoters and inhibitors) may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution, but differ as regards the arrangement of the atoms or groups in space.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The term “promoter” as used herein includes compounds that promote the expression or activity of a protein, polypeptide or enzyme and does not necessarily mean complete promotion of expression and/or activity. Rather, the promotion includes promotion of the expression and/or activity of a protein, polypeptide or enzyme to an extent, and for a time, sufficient to produce the desired effect.

The terms “calcify” and “calcification” refer to the accumulation of calcium salts in a tissue, in particularly in cancerous tumor tissue. These calcium salts include, for example, calcium phosphate, calcium carbonate, calcium oxalate, calcium pyrophosphate, hydroxyapatite, and combinations thereof. Calcification can be detected using imaging methods known in the art, for example, ultrasound, X-ray (including computed tomography (CT)), or magnetic resonance imaging (MRI).

The term “androgen receptor positive” cancer refers to a cancer that includes cancer cells that bind to androgens. Whether a particular cancer is androgen receptor positive can be determined using methods known in the art, for example, using an immunohistochemical assay performed using antibodies to androgen receptors.

The present disclosure is directed to methods of treating cancer in a patient comprising administering to the patient an effective amount of an agent that results in an increase in the amount of dihydrotestosterone (“DHT” or 5α-dihydrotestosterone or 5α-androstan-17β-ol-3-one) in the patient's blood. For example, exemplary aspects of the disclosure comprise administering to a patient an effective amount of DHT, a dihydrotestosterone derivative, a dihydrotestosterone promoter, or a combination thereof. According to the disclosure, the administration of dihydrotestosterone, a dihydrotestosterone derivative, a dihydrotestosterone promoter, or a combination thereof results in the treatment of the patient's cancer by slowing or stopping the progression of the cancer, by initiating the regression of the cancer, or by initiating remission of the cancer.

In some aspects, the methods of the disclosure comprise administering to the patient an effective amount of DHT.

In other aspects, the methods of the disclosure comprise administering to the patient an effective amount of a dihydrotestosterone derivative. Dihydrotestosterone derivatives are known in the art and include, for example, steroidal compounds including the following A-B-C-D core structure:

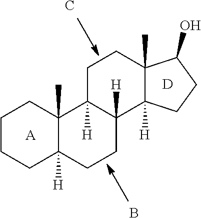

wherein the A-B-C-D core structure is substituted at any position with a substituent moiety, for example, a C₁₋₆ straight or branched alkyl moiety, an C₆₋₁₀ aryl moiety, or a 5- or 6- membered heteroaryl moiety that includes 1 or 2 heteroatoms selected from nitrogen, oxygen, and sulfur.

Dihydrotestosterone derivatives include, for example, mesterolone and drostanolone. A particularly preferred dihydrotestosterone derivative is stanozolol.

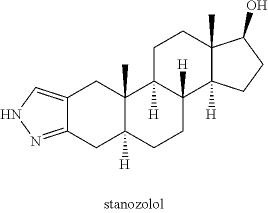

In other aspects, the methods of the disclosure comprise administering to the patient an effective amount of a dihydrotestosterone promoter. Dihydrotestosterone promoters are known in the art and include, for example, compounds that increase the amount of DHT in the patient's blood. Methods of detecting DHT in blood are known in the art.

The described methods and compositions can be used to treat cancer. For example, the cancers treated according to the methods described herein include, for example, non-small cell lung carcinoma, ovarian cancer, breast cancer, cervical cancer, pancreatic cancer, prostate cancer, stomach cancer, colon cancer, brain cancer, liver cancer, testicular cancer, leukemia, and lymphoma. The described methods are particularly effective in treating breast cancer.

In preferred aspects, the cancer is an androgen receptor positive cancer.

While not wishing to be bound to any particular theory, it is speculated that the administration of an effective amount of the dihydrotestosterone, the dihydrotestosterone derivative, the dihydrotestosterone promoter, or the combination thereof to the subject results in the calcification of the subject's cancerous tumor(s). The described methods are particularly effective in initiating calcification in cancers that are androgen positive cancers. In preferred methods, the effective amount of the dihydrotestosterone, the dihydrotestosterone derivative, the dihydrotestosterone promoter, or the combination thereof will produce calcification in at least a portion of the cancer. After the cancer has sufficiently calcified, for example, after at least about 20% of the tumor has calcified, the calcified tissue can be surgically excised using methods known in the art. In some aspects, the calcified tissue can be surgically excised after at least about 25%, 30%, 35%, 40%, 45%, 50%, 55%, 60%, 65%, 70%, 75%, 80%, 85%, 90%, 95%, or at least about 100% of the tumor has calcified.

According to the disclosure, the dihydrotestosterone, the dihydrotestosterone derivative, the dihydrotestosterone promoter, or the combination thereof can be administered to the patient orally, subcutaneously, intravenously, transdermally, vaginally, rectally, or in any combination thereof. In some aspects, dihydrotestosterone, a dihydrotestosterone derivative, a dihydrotestosterone promoter, or a combination thereof is administered orally. In other aspects, the dihydrotestosterone, dihydrotestosterone derivative, dihydrotestosterone promoter, or combination thereof is administered subcutaneously. In other aspects, the dihydrotestosterone, dihydrotestosterone derivative, dihydrotestosterone promoter, or combination thereof is administered intravenously. In other aspects, the dihydrotestosterone, dihydrotestosterone derivative, dihydrotestosterone promoter, or combination thereof is administered transdermally. In other aspects, the dihydrotestosterone, dihydrotestosterone derivative, dihydrotestosterone promoter, or combination thereof is administered vaginally. In other aspects, the dihydrotestosterone, dihydrotestosterone derivative, dihydrotestosterone promoter, or combination thereof is administered rectally. In some aspects, the dihydrotestosterone, dihydrotestosterone derivative, dihydrotestosterone promoter, or combination thereof is administered transdermally. In other aspects, the dihydrotestosterone, dihydrotestosterone derivative, dihydrotestosterone promoter, or combination thereof is administered orally.

In some aspects of the disclosure, the methods further comprise the administration of an effective amount of a tyrosine hydroxylase inhibitor, along with the effective amount of the dihydrotestosterone, dihydrotestosterone derivative, dihydrotestosterone promoter, or combination thereof. The tyrosine hydroxylase inhibitor can be administered simultaneously or at least contemporaneously with the dihydrotestosterone, dihydrotestosterone derivative, dihydrotestosterone promoter, or combination thereof. In other aspects, the tyrosine hydroxylase inhibitor is administered separately from the dihydrotestosterone, dihydrotestosterone derivative, dihydrotestosterone promoter, or combination thereof.

The tyrosine hydroxylase inhibitor can be a tyrosine derivative. The tyrosine derivative can be one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6- dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OME HCl, H-D-tyr-OMe HCl, D-tyrosine methyl ester hydrochloride, D-tyrosine-ome HCl, methyl D-tyrosinate hydrochloride, H-D-tyr-OMe·HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I2)-OSu, Fmoc-tyr(3-NO₂)-OH, and α-methyl-DL-tyrosine. A particularly preferred tyrosine hydroxylase inhibitor is a-methyl-DL-tyrosine.

According to the disclosure, the tyrosine hydroxylase inhibitor can be administered to the patient orally, subcutaneously, intravenously, transdermally, vaginally, rectally, or in any combination thereof. In some aspects, the tyrosine hydroxylase inhibitor is administered orally. In other aspects, the tyrosine hydroxylase inhibitor is administered subcutaneously. In other aspects, the tyrosine hydroxylase inhibitor is administered intravenously. In other aspects, the tyrosine hydroxylase inhibitor is administered transdermally. In other aspects, the tyrosine hydroxylase inhibitor is administered vaginally. In other aspects, the tyrosine hydroxylase inhibitor is administered rectally.

Those skilled in the art will be able to determine the therapeutically effective amount of the tyrosine hydroxylase inhibitor. For example, it is envisioned that about 10-2000 mg, preferably 150-300 mg, of the tyrosine hydroxylase inhibitor (e.g., α-methyl-DL-tyrosine) is orally administered daily. In some aspects, about 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg of the tyrosine hydroxylase inhibitor (e.g., α-methyl-DL-tyrosine) is administered daily. The daily dosages of the tyrosine hydroxylase inhibitor (e.g., α-methyl-DL-tyrosine) can be administered as a single dose or in substantially equal doses throughout the day. For example, the tyrosine hydroxylase inhibitor can be administered to the patient once a day, twice a day, three times a day, or four times a day.

In some aspects of the disclosure, the therapeutically effective amount of the dihydrotestosterone, dihydrotestosterone derivative, dihydrotestosterone promoter, or combination thereof (and the optional tyrosine hydroxylase inhibitor) is administered in combination with a therapeutically effective amount of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present disclosure are chemical compounds that increase the production and/or the activity of melanin. Melanin promoters are known in the art and include, for example, methoxsalen and melanotan II.

The melanin, a melanin promoter, or combination thereof can be administered simultaneously or at least contemporaneously with the dihydrotestosterone, dihydrotestosterone derivative, dihydrotestosterone promoter, or combination thereof (and the optional tyrosine hydroxylase inhibitor). In other aspects, the melanin, a melanin promoter, or combination thereof is administered separately from the dihydrotestosterone, dihydrotestosterone derivative, dihydrotestosterone promoter, or combination thereof (and the optional tyrosine hydroxylase inhibitor).

According to the disclosure, the melanin and/or melanin promoter can be administered to the patient orally, subcutaneously, intravenously, transdermally, vaginally, rectally, or in any combination thereof. In some aspects, the melanin and/or melanin promoter is administered orally. In other aspects, the melanin and/or melanin promoter is administered subcutaneously. In other aspects, the melanin and/or melanin promoter is administered intravenously. In other aspects, the melanin and/or melanin promoter is administered transdermally. In other aspects, the melanin and/or melanin promoter is administered vaginally. In other aspects, the melanin and/or melanin promoter is administered rectally.

Those skilled in the art will be able to determine the therapeutically effective amount of the melanin and/or melanin promoter. For example, it is envisioned that about 10-150 mcg of melanin, for example, about 10, 20, 30, 40, 50, 60, 70, 80, 90, 100, 110, 120, 130, 140 or about 150 mcg of melanin is orally administered daily. It is envisioned that 1-100 mg, for example, 1, 2, 3, 4, 5, 6, 7, 8, 9, 10, 15, 20, 25, 30, 35, 40, 45, 50, 55, 60, 65, 70, 75, 80, 85, 90, 95 or about 100 mg of a melanin promoter (e.g., methoxsalen or melanotan) is administered daily. The daily dosages of the melanin and/or melanin promoter can be administered as a single dose or in substantially equal doses throughout the day. For example, the melanin and/or melanin promoter can be administered to the patient once a day, twice a day, three times a day, or four times a day.

In some aspects of the disclosure, the therapeutically effective amount of dihydrotestosterone, dihydrotestosterone derivative, dihydrotestosterone promoter, or combination thereof (and the optional tyrosine hydroxylase inhibitor, melanin, and/or melanin promoter) is administered in combination with a therapeutically effective amount of a p450 3A4 promoter. “Cytochrome p450 3A4” (which can be abbreviated as “p450 3A4”) is a member of the cytochrome p450 superfamily of enzymes and is a mixed-function oxidase that is involved in the metabolism of xenobiotics in the body. p450 3A4 promoters are known in the art and include, for example, 5,5-diphenylhydantoin, valproic acid, and carbamazepine.

According to the disclosure, the p450 3A4 promoter can be administered to the patient orally, subcutaneously, intravenously, transdermally, vaginally, rectally, or in any combination thereof. In some aspects, the p450 3A4 promoter is administered orally. In other aspects, the p450 3A4 promoter is administered subcutaneously. In other aspects, the p450 3A4 promoter is administered intravenously. In other aspects, the p450 3A4 promoter is administered transdermally. In other aspects, the p450 3A4 promoter is administered vaginally. In other aspects, the p450 3A4 promoter is administered rectally.

Those skilled in the art will be able to determine the therapeutically effective amount of the p450 3A4 promoter. For example, it is envisioned that about 1-100 mg of the p450 3A4 promoter (e.g., 5,5-diphenylhydantoin, valproic acid, or carbamazepine), for example, about 1, 2, 3, 4, 5, 6, 7, 8, 9, 10, 15, 20, 25, 30, 35, 40, 45, 50, 55, 60, 65, 70, 75, 80, 85, 90, 95 or about 100 mg of the p450 3A4 promoter (e.g., 5,5-diphenylhydantoin, valproic acid, or carbamazepine) is administered daily. The daily dosages of the p450 3A4 promoter can be administered as a single dose or in substantially equal doses throughout the day. For example, the p450 3A4 promoter can be administered to the patient once a day, twice a day, three times a day, or four times a day.

The p450 3A4 promoter can be administered simultaneously or at least contemporaneously with the dihydrotestosterone, dihydrotestosterone derivative, dihydrotestosterone promoter, or combination thereof (and the optional tyrosine hydroxylase inhibitor, melanin, and/or melanin promoter). In other aspects, p450 3A4 promoter is administered separately from the dihydrotestosterone, dihydrotestosterone derivative, dihydrotestosterone promoter, or combination thereof (and the optional tyrosine hydroxylase inhibitor, melanin, and/or melanin promoter).

In some aspects of the disclosure, the therapeutically effective amount of dihydrotestosterone, a dihydrotestosterone derivative, dihydrotestosterone promoter, or combination thereof (and the optional tyrosine hydroxylase inhibitor, melanin, melanin promoter, and/or p450 3A4 promoter) is administered in combination with a therapeutically effective amount of a growth hormone inhibitor. Growth hormones (such as, for example, pancreatic growth hormone) induce cell replication. Growth hormone inhibitors are known in the art and include, for example, octreotide, somatostatin, and seglitide.

According to the disclosure, the growth hormone inhibitor can be administered to the patient orally, subcutaneously, intravenously, transdermally, vaginally, rectally, or in any combination thereof. In some aspects, the growth hormone inhibitor is administered orally. In other aspects, the growth hormone inhibitor is administered subcutaneously. In other aspects, the growth hormone inhibitor is administered intravenously. In other aspects, the growth hormone inhibitor is administered transdermally. In other aspects, the growth hormone inhibitor is administered vaginally. In other aspects, the growth hormone inhibitor is administered rectally.

Those skilled in the art will be able to determine the therapeutically effective amount of the growth hormone inhibitor. For example, it is envisioned that about 1 mcg -100 mg of the growth hormone inhibitor is administered orally, subcutaneously, or intravenously daily. The daily dosages of the growth hormone inhibitor can be administered as a single dose or in substantially equal doses throughout the day. For example, the growth hormone inhibitor can be administered to the patient once a day, twice a day, three times a day, or four times a day.

In some aspects of the disclosure, the therapeutically effective amount of dihydrotestosterone, dihydrotestosterone derivative, dihydrotestosterone promoter, or combination thereof (and the optional tyrosine hydroxylase inhibitor, melanin, melanin promoter, p450 3A4 promoter, and/or growth hormone inhibitor) is administered in combination with a therapeutically effective amount of D-leucine. D-leucine is believed to create a physiological environment that mimics a leucine shortage.

According to the disclosure, the D-leucine can be administered to the patient orally, subcutaneously, intravenously, transdermally, vaginally, rectally, or in any combination thereof. In some aspects, the D-leucine is administered orally. In other aspects, the growth hormone inhibitor is administered subcutaneously. In other aspects, the D-leucine is administered intravenously. In other aspects, the D-leucine is administered transdermally. In other aspects, the D-leucine is administered vaginally. In other aspects, the D-leucine is administered rectally.

Those skilled in the art will be able to determine the therapeutically effective amount of the D-leucine. For example, it is envisioned that about 1-2000 mg of the D-leucine is administered orally daily. The daily dosages of the D-leucine can be administered as a single dose or in substantially equal doses throughout the day. For example, the D-leucine can be administered to the patient once a day, twice a day, three times a day, or four times a day.

In preferred aspects of the disclosure, the patient's cancer is assessed prior to the administration of the dihydrotestosterone, dihydrotestosterone derivative, dihydrotestosterone promoter, or combination thereof to determine the cancer's stage (and the optional tyrosine hydroxylase inhibitor, melanin, melanin promoter, p450 3A4 promoter, growth hormone inhibitor, and/or D-leucine). In other preferred aspects, the patient's cancer is assessed after the administration of the dihydrotestosterone, dihydrotestosterone derivative, dihydrotestosterone promoter, or combination thereof (and the optional tyrosine hydroxylase inhibitor, melanin, melanin promoter, p450 3A4 promoter, growth hormone inhibitor, and/or D-leucine) to determine the cancer's progression or regression.

Also provided herein are kits for use in the described methods. Kits of the disclosure will include dihydrotestosterone, a dihydrotestosterone derivative, a dihydrotestosterone promoter, or a combination thereof (e.g., N-[(2S,3R)-3-amino-2-hydroxy-4-phenylbutyryl]-L-leucine or rapamycin), together with packaging for same. The kits can optionally include a tyrosine hydroxylase inhibitor (e.g., α-methyl-DL-tyrosine), melanin and/or a melanin promoter (e.g., melanin, methoxsalen, and/or melanotan II), a p450 3A4 promoter (e.g., 5,5-diphenylhydantoin, valproic acid, or carbamazepine), a leucine aminopeptidase inhibitor (e.g., rapamycin and/or N-[(2S,3R)-3-amino-2-hydroxy-4-phenylbutyryl]-L-leucine), a growth hormone inhibitor (e.g., pancreatic growth hormone inhibitor, somatostatin, or octreotide) and/or D-leucine, together with packaging for same. The kit can include one or more separate containers, dividers or compartments and, optionally, informational material such as instructions for administration. For example, each inhibitor or promoter (or the various combinations thereof) can be contained in a bottle, vial, or syringe, and the informational material can be contained in a plastic sleeve or packet or provided in a label. In some embodiments, the kit includes a plurality (e.g., a pack) of individual containers, each containing one or more unit dosage forms of a compound described herein. For example, the kit can include a plurality of syringes, ampules, foil packets, or blister packs, each containing a single unit dose of a compound described herein or any of the various combinations thereof. The containers of the kits can be air tight, waterproof (e.g., impermeable to changes in moisture or evaporation), and/or light-tight. The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

Also provided are pharmaceutical compositions for use with the described methods. The pharmaceutical compositions will comprise any combination of the described active agents in combination with one or more pharmaceutically acceptable excipients. Pharmaceutically acceptable excipients are known in the art. See, e.g., Remington's 17th Edition Pharmaceutical Sciences, Mack Publishing Company (1985).

The pharmaceutical compositions of the disclosure can include dihydrotestosterone, a dihydrotestosterone derivative, a dihydrotestosterone promoter, or a combination thereof in combination with a tyrosine hydroxylase inhibitor; melanin, a melanin promotor, or a combination thereof a p450 3A4 promoter, or a combination thereof. Other pharmaceutical compositions may further comprise a growth hormone inhibitor such as pancreatic growth hormone inhibitor, octreotide, or somatostatin. Further pharmaceutical compositions may further comprise D-leucine.

In some aspects, the pharmaceutical compositions can include a combination of dihydrotestosterone, a dihydrotestosterone derivative, a dihydrotestosterone promoter, or a combination thereof, and a tyrosine hydroxylase inhibitor such as, for example, α-methyl-DL-tyrosine.

In some aspects, the pharmaceutical compositions can include a combination of dihydrotestosterone, a dihydrotestosterone derivative, a dihydrotestosterone promoter, or a combination thereof, and melanin, a melanin promoter, or a combination thereof. For example, the pharmaceutical compositions can include a combination of dihydrotestosterone, a dihydrotestosterone derivative, a dihydrotestosterone promoter, or a combination thereof, a leucine aminopeptidase inhibitor, and melanin, methoxsalen, melanotan II, or a combination thereof.

In some aspects, the pharmaceutical compositions can include a combination of dihydrotestosterone, a dihydrotestosterone derivative, a dihydrotestosterone promoter, or a combination thereof, and a p450 3A4 promoter. For example, the pharmaceutical compositions can include a combination of dihydrotestosterone, a dihydrotestosterone derivative, a dihydrotestosterone promoter, or a combination thereof, and 5,5-diphenylhydantoin, valproic acid, or carbamazepine.

In some aspects, the pharmaceutical compositions can include a combination of dihydrotestosterone, a dihydrotestosterone derivative, a dihydrotestosterone promoter, or a combination thereof, and a growth hormone inhibitor such as, for example, pancreatic growth hormone, octreotide, or somatostatin.

In some aspects, the pharmaceutical compositions can include a combination of dihydrotestosterone, a dihydrotestosterone derivative, a dihydrotestosterone promoter, or a combination thereof, and D-leucine.

As described herein, certain preferred methods of the disclosure include the transdermal administration of any of the described active agents. For example, in some aspects, the dihydrotestosterone, a dihydrotestosterone derivative, a dihydrotestosterone promoter, or a combination thereof, and optionally with any of the other active agents described herein, is administered transdermally. The active agents can be transdermally administered in the same transdermal formulation. Alternatively, the active agents can be administered in separate transdermal formulations. Transdermal formulations are known in the art. Preferred formulations include those described in, for example, International Application No. PCT/US2015/000302, filed Dec. 23, 2015, the entirety of which is incorporated by reference herein. For example, suitable transdermal formulations for use with any of the described methods can include nonaethylene glycol monododecyl ether, 1-methyl-2-pyrrolidinone, ethanol, and oleic acid, in combination with any of the described active agents. Other suitable transdermal formulations for use with any of the described methods can include nonaethylene glycol monododecyl ether, 1-methyl-2-pyrrolidinone, ethanol, and linoleic acid, in combination with any of the described active agents.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1—Methods of Treating Cancer

Patients are screened for cancer, for example non-small cell lung carcinoma, ovarian cancer, breast cancer, cervical cancer, pancreatic cancer, prostate cancer, stomach cancer, colon cancer, brain cancer, liver cancer, testicular cancer, leukemia, and lymphoma. DHT, a dihydrotestosterone derivative, a dihydrotestosterone promoter, or a combination thereof, is administered to each patient in an amount and for a time sufficient to achieve a therapeutic effect. Preferably, the patients are administered the dihydrotestosterone derivative, stanozolol. The methods can optionally include the administration of an effective amount of a tyrosine hydroxylase inhibitor (e.g., α-methyl-DL-tyrosine); an effective amount of melanin, a melanin promoter, or a combination thereof (e.g., melanin, methoxsalen, or melanotan II); an effective amount of a p450 3A4 promoter (e.g., 5, 5-diphenylhydantoin, valproic acid, or carbamazepine); an effective amount of a growth hormone inhibitor e.g., pancreatic growth hormone inhibitor, octreotide, somatostatin); an effective amount of D-leucine; and any combination thereof.

### Example 2—Method of Treating Cancer

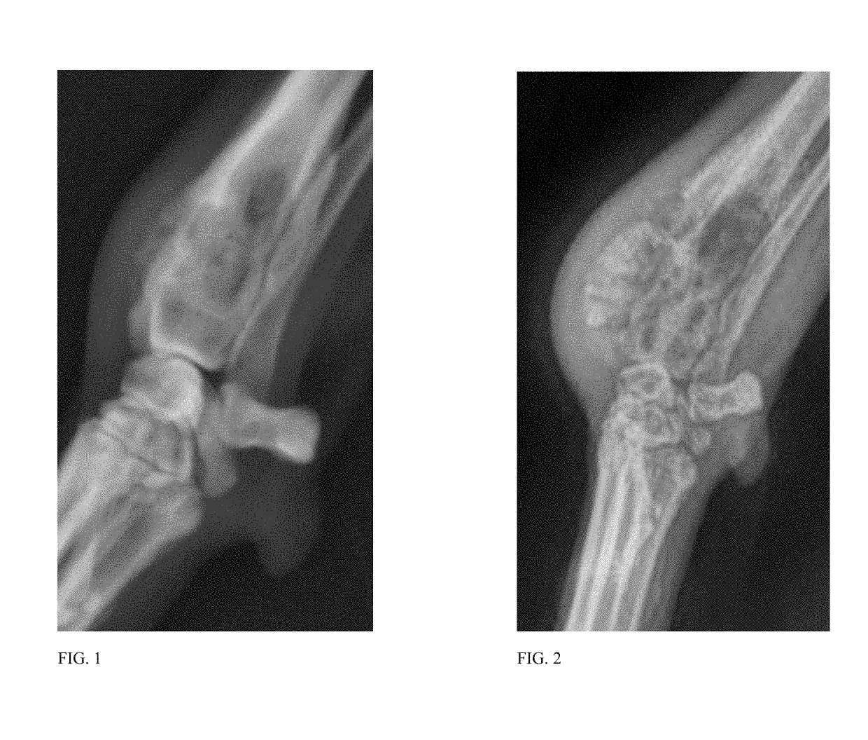

A canine presenting with a cancerous tumor of the distal radius (see FIG. 1) was administered stanozolol. After stanozolol administration, the cancerous tumor calcified (see FIG. 2).

1. A method of treating cancer in a patient comprising administering to the patient an effective amount of dihydrotestosterone, a dihydrotestosterone derivative, a dihydrotestosterone promoter, or a combination thereof.

2. The method of claim 1, wherein the amount of the dihydrotestosterone, a dihydrotestosterone derivative, a dihydrotestosterone promoter, or a combination thereof is sufficient to produce calcification of at least a portion of the cancer.

3. The method of claim 1, wherein the dihydrotestosterone derivative is stanozolol.

4. The method of claim 1, further comprising administering an effective amount of a tyrosine hydroxylase inhibitor.

5. The method of claim 1, further comprising administering an effective amount of melanin, a melanin promoter, or a combination thereof; a p450 3A4 promoter; a leucine aminopeptidase inhibitor; or a combination thereof.

6. The method of claim 1, further comprising administering a growth hormone inhibitor.

7. The method of claim 5, further comprising administering a growth hormone inhibitor.

8. The method of claim 4, wherein the tyrosine hydroxylase inhibitor is methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6- dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, H-D-tyr-OMe·HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)-OSu, Fmoc-tyr(3-NO₂)-OH, α-methyl-DL-tyrosine, or a combination thereof.

9. The method of claim 5, wherein the melanin promoter is methoxsalen or melanotan II.

10. The method of claim 5, wherein the p450 3A4 promoter is 5,5-diphenylhydantoin, valproic acid, or carbamazepine

11. The method of claim 5, wherein the leucine aminopeptidase inhibitor is N-[(2S,3R)-3-amino-2-hydroxy-4-phenylbutyryl]-L-leucine.

12. The method of claim 5, wherein the leucine aminopeptidase inhibitor is rapamycin.

13. The method of claim 6, wherein the growth hormone inhibitor is a pancreatic growth hormone inhibitor.

14. The method of claim 6, wherein the growth hormone inhibitor is octreotide or somatostatin.

15. The method of claim 7, wherein the growth hormone inhibitor is a pancreatic growth hormone inhibitor.

16. The method of claim 7, wherein the growth hormone inhibitor is octreotide or somatostatin.

17. The method of claim 1, wherein the administration of any of the therapeutic agents is oral, subcutaneous, intravenous, transdermal, vaginal, rectal, or in any combination thereof.

18. The method of claim 1, wherein the cancer is an androgen receptor positive cancer.

19. The method of claim 1, wherein the cancer is non-small cell lung carcinoma, ovarian cancer, breast cancer, cervical cancer, pancreatic cancer, prostate cancer, stomach cancer, colon cancer, brain cancer, liver cancer, testicular cancer, leukemia, or lymphoma.

20. The method of claim 2, further comprising surgically excising the calcified cancer from the patient.

21. The method of claim 4, wherein the tyrosine hydroxylase inhibitor comprises α-methyl-DL-tyrosine.

22. The method of claim 1, wherein the cancer is breast cancer

23. The method of claim 4, wherein the dihydrotestosterone derivative is stanozolol, the tyrosine hydroxylase inhibitor comprises a-methyl-DL-tyrosine, and the cancer is breast cancer.
